(19) United States
(12) Patent Application Publication
(10) Pub. No.: US 2015/0152032 A1
(43) Pub. Date: Jun. 4, 2015

(54) BUTANOL RECOVERY PROCESS

(71) Applicant: INVISTA NORTH AMERICA S.A.R.L., Wilmington, DE (US)

(72) Inventors: Graham R. Aird, Darlington (GB); Colin Anderson, Aberdeen (GB); Rakhesh Martyn, Yarm (GB); Philip N. Ward, Darlington (GB); Ashley Wells, Yarm (GB); Robert E. Stilson, Wilmington, NC (US)

(73) Assignee: INVISTA NORTH AMERICA S.A.R.L., Wilmington, DE (US)

(21) Appl. No.: 14/406,898

(22) PCT Filed: Jun. 4, 2013
(86) PCT No.: PCT/US2013/043987

§ 371 Date: Dec. 10, 2014

(30) Foreign Application Priority Data
Jun. 11, 2012 (GB) 1210256.2

(51) Int. Cl. C07C 29/82; B01D 3/14; B01D 3/10

(52) U.S. Cl. CPC C07C 29/82; B01D 3/10; B01D 3/143

(57) ABSTRACT

The present invention provides an improved process for recovering butanol from a mixture comprising water, methanol, propanol, butanol and optionally other organic compounds. More particularly, the invention relates to a process for recovering butanol as an essentially pure product from a mixture comprising water, methanol, propanol, butanol and other organic compounds.

### CROSS-REFERENCE TO RELATED APPLICATION

This application claims benefit of priority from Great Britain Application No. 1210256.2 filed Jun. 11, 2012. This application hereby incorporates by reference this application in its entirety.

### FIELD OF THE INVENTION

The present invention relates to an improved process for recovering butanol from a mixture comprising water, methanol, propanol, butanol and, optionally, other organic species providing undesirable color. More particularly, the invention relates to a process for recovering butanol as a pure product (e.g., from about 95 to 100 wt. % butanol) from a mixture comprising water, methanol, propanol, butanol and optionally other organic compounds providing undesirable color.

### BACKGROUND OF THE INVENTION

In processes for converting, for example, acetylene to butanediol, a number of side products are produced in addition to the butanediol. These side products, which are difficult to separate, comprise water, methanol, propanol, butanol and various organic species providing undesirable color. SRI of IHS, Inc. (321 Inverness Drive South, Englewood, Colo. 80112, U.S.A.) in its Process Economics Report 96B, titled “Polybutylene Terephthalate (PBT) and Butanediol” (see http://chemical.ihs.com/PEP/public/reports/phase_{—}95/rp096B/) shows a process in which the above alcohols and other organic species are separated from water in a distillation column and remain in the column overhead. The overhead stream will contain 3 to 70 wt. % water and 30 to 97 wt. % organics. At the present time, this overhead is treated as a waste stream; however the butanol (the highest concentration of the organics) would be valuable as a purified product.

Canada Patent No. 1,706,165A discloses a process for separating tertiary butyl alcohol from mixture with water by heterogeneous azeotropic rectifying. The process system comprises a rectifying tower and includes proportionally adding the mixture and cyclohexane to the rectifying tower. Phase separation is also included with tertiary butyl alcohol recovered from the top of the tower.

U.S. Pat. No. 5,625,109A discloses a process whereby a tertiary butyl alcohol feed is continuously dehydrated in the liquid phase. In a boiling reactor, a vapor mixture of isobutylene, water, tertiary butyl alcohol and isobutanol and/or secondary butanol is removed and cooled, isobutylene is recovered and the remaining product mixture is phase separated into an upper organic-rich phase which is recycled to dehydration and a lower aqueous phase which is purged.

U.S. Pat. No. 5,449,440A discloses a three distillation column process for forming three separate streams from a feed mixture containing methanol, ethanol, n-propanol, isobutanol, water and other high-boiling and low-boiling compounds. The streams said to be formed are an anhydrous stream of methanol or methanol and ethanol, one containing most of the n-propanol present in the feed mixture and one containing most of the isobutanol present in the feed mixture. The latter stream is exemplified to comprise about 62% isobutanol.

France Patent No. 2,383,688A1 discloses a process for separation of acetic acid, n-butanol, ethanol, propanol or methanol from mixture with water by azeotropic distillation.

The above methods for separating compounds such as butanol from mixture with water can add complexity, processing limitations, and/or cost to the process. A simple economical process for recovering butanol, especially in high purity, from a mixture comprising water, methanol, propanol, butanol and a range of organic species displaying undesirable color is needed.

### SUMMARY OF THE INVENTION

The present invention provides a simple economical process for recovering butanol in high purity from a mixture comprising water, methanol, propanol, butanol and, optionally, a range of other organic compounds, such as those displaying or providing undesirable color. The process involves a particular arrangement of three to four distillation columns, hereinafter more particularly described, and one to three phase separators, hereinafter more particularly described. An embodiment of the process, involving three distillation columns and one or two phase separators, comprises the steps of:

a) feeding a mixture comprising water, methanol, propanol, butanol and other organic compounds, such as those having or providing an undesirable color factor, hereinafter more particularly described, to a first distillation column maintained at controlled conditions, hereinafter more particularly described, via a side inlet whereby a bottom fraction stream of the first distillation column comprises water, and a top fraction stream of the first distillation column comprises methanol, propanol, butanol, remaining water (azeotrope) and other organic compounds,

b) optionally feeding the top fraction stream of step a) to a first phase separator, whereby a bottom stream of the first phase separator comprises water, and a top stream of the first phase separator comprises methanol, propanol, butanol, remaining water (azeotrope) and other organic compounds,

c) feeding the top fraction stream of step a) or top stream of step b) to a second distillation column maintained at controlled conditions, hereinafter more particularly described, via a side inlet, whereby a side fraction stream of the second distillation column comprises propanol, butanol, water (azeotrope) and other organic compounds, a top fraction stream of the second distillation column comprises methanol, propanol, volatile organic compounds and water, and a bottom fraction stream of the second distillation column comprises butanol and nonvolatile organic compounds,

d) feeding said side fraction stream of step c) to a second phase separator, whereby a top organic phase stream and a bottom aqueous phase stream are created,

e) feeding said organic phase stream of step d) to the second distillation column below the outlet for the side fraction stream of the second distillation column,

f) feeding said aqueous phase stream of step d) and bottom stream of step b) to the first distillation column (optionally via the first phase separator),

g) feeding said bottom fraction stream from step c) comprising butanol and nonvolatile organic compounds to a third distillation column maintained at controlled conditions, hereinafter more particularly described, whereby a bottom fraction stream of the third distillation column comprises butanol and nonvolatile organic compounds, and a top fraction stream of the third distillation column comprises from 95 to about 100 wt. % butanol, and
h) recovering said butanol from the top fraction stream of step g).

An embodiment of the present invention, involving four distillation columns and three phase separators, comprises the steps of:

a) feeding a mixture comprising water, methanol, propanol, butanol and other organic compounds, such as those having or providing an undesirable color factor, hereinafter more particularly described, to a first distillation column maintained at controlled conditions, hereinafter more particularly described, via a side inlet whereby a bottom fraction stream of the first distillation column comprises water, and a top fraction stream of the first distillation column comprises methanol, propanol, butanol, remaining water (azeotrope) and other organic compounds,

b) feeding the top fraction stream of step a) to a first phase separator, whereby a bottom aqueous phase stream and a top organic phase stream are created,

c) feeding said aqueous phase stream of step b) to the first distillation column,

d) feeding said organic phase stream of step b) to a second distillation column maintained at controlled conditions, hereinafter more particularly described, via a side inlet whereby a bottom fraction stream of the second distillation column comprises propanol, butanol, water (azeotrope) and nonvolatile organic compounds, and a top fraction stream of the second distillation column comprises methanol and volatile organic compounds,

e) feeding the bottom fraction stream of step d) to a second phase separator, whereby a bottom aqueous phase stream and a top organic phase stream are created,

f) feeding said aqueous phase stream of step e) to the first distillation column (optionally via the first phase separator),

g) feeding said organic phase stream of step e) to a third distillation column maintained at controlled conditions, hereinafter more particularly described, via a side inlet, whereby a side fraction stream of the third distillation column comprises propanol, butanol, water (azeotrope) and other organic compounds, a top fraction stream of the third distillation column comprises methanol, propanol, volatile organic compounds and water and a bottom fraction stream of the third distillation column comprises butanol and nonvolatile organic compounds,

h) feeding the side fraction stream of step g) to a third phase separator, whereby a bottom aqueous phase stream and a top organic phase stream are created,

i) feeding the top organic phase stream of step h) via a side inlet to the third distillation column, optionally at a point below that from which the side fraction is drawn,

j) feeding the bottom aqueous phase stream of step h) to the first distillation column (optionally via the first phase separator),

k) feeding said bottom fraction stream of step g) comprising butanol and nonvolatile organic compounds to a fourth distillation column maintained at controlled conditions, hereinafter more particularly described, whereby a bottom fraction stream of the fourth distillation column comprises butanol and nonvolatile organic compounds, and a top fraction stream of the fourth distillation column comprises from 95 to about 100 wt. % butanol, and

l) recovering said butanol from the top fraction stream of step k).

### BRIEF DESCRIPTION OF THE DRAWINGS

FIG. 1 shows a diagrammatic flow of an embodiment of the present process involving three distillation columns and two phase separators.

FIG. 2 shows a diagrammatic flow of another embodiment of the present process involving four distillation columns and three phase separators.

### DETAILED DESCRIPTION OF THE INVENTION

As a result of intense research in view of the above, we have found that we can economically and effectively recover butanol in high purity from a mixture comprising water, methanol, propanol, butanol and, optionally, other organic compounds, such as those having or providing an undesirable color factor. The process involves particular arrangements of three to four distillation columns and one to three phase separators. One embodiment of the process of the invention comprises a particular arrangement of three distillation columns and two phase separators, one of which is optional, and another embodiment, four distillation columns and three phase separators, to separate and recover high value butanol in high purity from a mixture which is difficult to separate comprising water, methanol, propanol, butanol and optionally other organic compounds.

The term “high purity”, as used herein, means from about 95 to 100 wt. %, such as at least about 96 wt. %, such as at least about 99 wt. %, for example 99.5 wt. % or higher. The term “butanol”, as used herein, means all isomers thereof, e.g. n-butyl alcohol, sec-butyl alcohol and isobutyl alcohol. The term “propanol”, as used herein, means all isomers thereof, e.g. n-propyl alcohol and isopropyl alcohol. The term “color factor”, as used herein, means the “platinum-cobalt” color scale value determined by ASTM D1209, Standard Test Method for Color of Clear Liquids. The term “volatile”, as used herein, means that the boiling point of the compound(s) to which it refers is less than 117° C. The term “nonvolatile”, as used herein, means that the boiling point of the compound(s) to which it refers is 117° C. or higher.

The distillation columns for use in the present invention may comprise either packing or trays to provide mass transfer, however, those columns in which two liquid phases are present may preferably use trays to ensure better liquid mixing.

The phase separators for use in the present invention comprise simple decanters and may also comprise coalescers. Phase separators may be either horizontally or vertically oriented and must be designed with sufficient residence time and cross-sectional area to enable adequate time for phase separation to occur. A coalescer can advantageously be used to enhance the coalescence of droplets to facilitate phase separation. The coalescer may consist of wire-wool, corrugated sheets or other such common designs for such devices.

An embodiment of the feed mixture to be separated by the process of the present invention comprises from about 50 to about 100%, e.g. 50 to 99.7%, water, from about 0.1 to about 15% methanol, from about 0.1 to about 5% propanol, and from about 0.1 to about 30% butanol. Other extraneous components of the mixture, if any, will be small amounts of other organic compounds, such as those having or providing an undesirable color factor. It is determined that a color factor of greater than 15, for example greater than 20, is undesirable; and that a color factor less than 15 is desirable. Preferably, the butanol recovered from the processes of the present invention has a color factor less than 15.

In the processes of the present invention, the distillation columns are maintained under controlled conditions. With regard to the first distillation column, this may be operated at a pressure in the range from 0.1 to 450 bara, alternatively in the range from 0.2 to 3 bara. The first distillation column may have a tops temperature in the range from 30 to 250° C. and/or a bottoms temperature in the range from 40 to 270° C. In one embodiment, the first distillation column is maintained at a pressure in the range from 0.1 to 450 bara, with a tops temperature in the range from 30 to 250° C. and a bottoms temperature in the range from 40 to 270° C.

The second distillation column may be operated at a pressure in the range from 0.1 to 35 bara, alternatively in the range from 0.2 to 2 bara. The second distillation column may have a tops temperature in the range from 30 to 250° C. and/or a bottoms temperature in the range from 60 to 270° C. In one embodiment, the second distillation column is maintained at a pressure in the range from 0.1 to 35 bara, with a tops temperature in the range from 30 to 250° C. and a bottoms temperature in the range from 60 to 270° C. These conditions may, in particular, be used for the second distillation column where the process involves three distillation columns and one or two phase separators as described herein.

In an alternative embodiment, the second distillation column may be operated at a pressure in the range from pressure in the range from 0.2 to 50 bara, alternatively in the range from 0.2 to 2 bara. The second distillation column may have a tops temperature in the range from 30 to 200° C. and/or a bottoms temperature in the range from 55 to 270° C. In one embodiment, the second distillation column is maintained at a pressure in the range from 0.2 to 50 bara, with a tops temperature in the range from 30 to 200° C. and a bottoms temperature in the range from 55 to 270° C. These conditions may, in particular, be used for the second distillation column where the process involves four distillation columns and three phase separators as described herein.

The third distillation column may be operated at a pressure in the range from 0.01 to 35 bara, alternatively in the range from 0.1 to 35 bara, alternatively in the range from 0.1 to 2 bara. In particular, where the process of the invention involves three distillation columns and one or two phase separators, the third distillation column may be maintained at a pressure in the range from 0.01 to 35 bara. In one embodiment, in particular where the process of the invention involves four distillation columns and three phase separators, the third distillation column may be maintained at a pressure in the range from 0.1 to 35 bara, alternatively, 0.1 to 2 bara.

Where present, the fourth distillation column may be operated at a pressure in the range from 0.02 to 35 bara, in one embodiment, 0.1 to 2 bara.

In one embodiment wherein the process involves three distillation columns and one or two phase separators, the controlled conditions of the first distillation column include a pressure from 0.1 to 450 bara, the controlled conditions of the second distillation column include a pressure from 0.1 to 35 bara and the controlled conditions of the third distillation column include a pressure from 0.01 to 35 bara.

In an alternative embodiment wherein the process involves four distillation columns and three phase separators, the controlled conditions of the first distillation column include a pressure from 0.1 to 450 bara, the controlled conditions of the second distillation column include a pressure from 0.2 to 50 bara, the controlled conditions of the third distillation column include a pressure from 0.1 to 35 bara and the controlled conditions of the fourth distillation column include a pressure from 0.02 to 35 bara.

In an alternative embodiment wherein the process involves four distillation columns and three phase separators, the controlled conditions of the first distillation column include a pressure from 0.2 to 3 bara, the controlled conditions of the second distillation column include a pressure from 0.2 to 2 bara, the controlled conditions of the third distillation column include a pressure from 0.1 to 2 bara and the controlled conditions of the fourth distillation column include a pressure from 0.1 to 2 bara.

In a process of the present invention which includes three distillation columns and one or two phase separators, the side fraction stream draw-off location of step c) may be below the feed location of step c). Where this is the case, the side fraction stream may be maintained at a temperature less than 100° C.

In a process of the present invention which includes four distillation columns and three phase separators, the side fraction stream draw-off location of step g) may be below the feed location of step g) on the third distillation column. Where this is the case, the side fraction stream may be maintained at a temperature of less than 100° C.

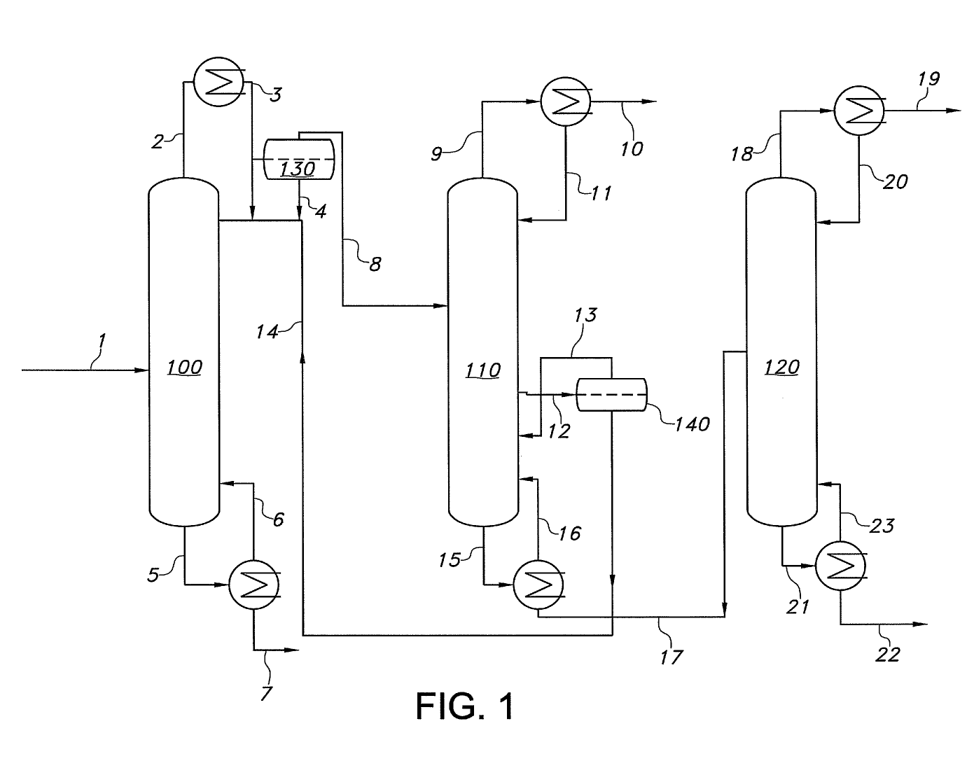

Referring more particularly to the drawings, where conditions are presented as non-limiting examples, FIG. 1 shows an embodiment of the present invention with the particular arrangement of three distillation columns and two phase separators. In this embodiment, feedstock comprising, for example, 6% methanol, 1% propanol, 4% butanol, 89% water and traces of other organic compounds providing an undesirable color factor, enters distillation column 100 via stream 1. Column 100 is maintained at a pressure from 0.1 to 450 bara with tops temperature from 30 to 250° C. and bottoms temperature from 40 to 270° C. Stream 5 feeds a reboiler from which stream 6 is returned to column 100 as a vapor stream, and stream 7 comprising principally water is drawn off from the bottom of column 100. A stream 2 comprising methanol, propanol, butanol, remaining water (azeotrope) and other organic compounds is condensed and drawn off the top as stream 3. Stream 3, having a color factor greater than 15, is fed to phase separator 130 maintained at a temperature of from 20 to 100° C. and pressure of from 0.1 to 450 bara. Aqueous stream 4 from phase separator 130 is returned to distillation column 100 as reflux. Organic stream 8 from phase separator 130 is fed to distillation column 110, which is maintained at a pressure from 0.1 to 35 bara with tops temperature from 30 to 250° C. and bottoms temperature from 60 to 270° C. Stream 3 can be refluxed directly to column 100 for control purposes if desired.

A two-phase mixture forms below the feed point in column 110 resulting from the predominant presence of a butanol-water azeotropic mixture. A side draw stream 12 from column 110 at less than 100° C. and comprising mainly propanol, butanol and water is fed to phase separator 140 to remove additional water from the system via aqueous stream 14 which is recycled to the column 100, e.g. with stream 4. The organic stream 13 (now significantly reduced in water concentration) from separator 140 is recycled to column 110 below the side draw for stream 12. The residual water in column 110 co-distills from the column top as stream 9 with the remaining propanol along with methanol. This stream is condensed; a portion refluxed as stream 11 and the balance is removed from the top of column 110 via stream 10. Any residual water will form an azeotrope with butanol and will be removed from column 110 via stream 10, but this is a very low loss of butanol. Stream 15 feeds a reboiler from which vapor is returned to the column via stream 16, while the remaining butanol, depleted of water, is removed from the column 110 bottom with other nonvolatile organic compounds via stream 17. Stream 17, having a color factor greater than 15, is fed to column 120, operating at from 0.01 to 35 bara, to distill clear, purified butanol having an improved color factor, e.g. less than 15, over the top via stream 18. This stream is condensed and a portion refluxed via stream 20 and the balance removed via stream 19. Stream 21 feeds a reboiler from which vapor is returned to the column via stream 23, while the remaining residue, having a color factor greater than 30 and comprising nonvolatile organic compounds, is removed from the bottom of the column via stream 22.

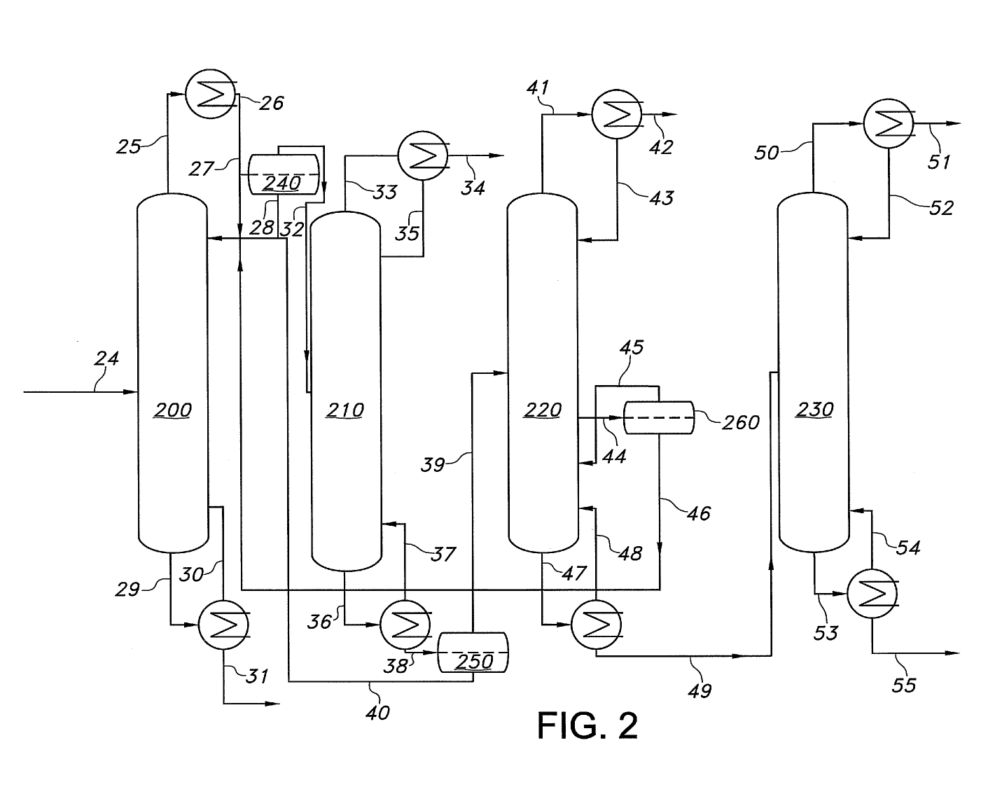

FIG. 2 shows another embodiment of the present invention with the particular arrangement of four distillation columns and three phase separators. In this embodiment, feedstock comprising, for example, 6% methanol, 1% propanol, 4% butanol, 89% water and traces of other organic compounds providing an undesirable color factor, enters distillation column 200 via steam 24. Column 200 (having similar duty to that of column 100 in FIG. 1) is maintained at a pressure from 0.1 to 450 bara. Bottoms stream 29 feeds a reboiler from which vapor is returned to column 200 via stream 30. The remaining liquor, comprising principally water, is removed from the bottom of the column via stream 31. Stream 25, comprising methanol, propanol, butanol, remaining water (azeotrope) and other organic compounds, is taken from the top of column 200, condensed to become liquid stream 26 and is optionally fed to separator 240. From separator 240 an aqueous bottom stream 28 is recycled to column 200, optionally as reflux. Top stream 32, comprising methanol, propanol, butanol, remaining water (azeotrope) and other organic compounds, is fed to distillation column 210, which is maintained at a pressure from 0.2 to 50 bara with tops temperature from 30 to 200° C. and bottoms temperature from 55 to 270° C. The tops condensate from column 200 can optionally be refluxed directly to the column via stream 27 rather than phase separated if desired.

From column 210, a top stream 33 comprising principally methanol and other volatile organic compounds is condensed and a portion refluxed via stream 35 with the remainder drawn off via stream 34. Stream 34 may be directed to a methanol recovery system. A bottoms stream 36 from column 210 is fed to the column reboiler from which vapor stream 37 returns to the column and bottoms stream 38, comprising butanol, propanol, any remaining water (azeotrope) and nonvolatile organic compounds, is optionally fed to separator 250, resulting in an aqueous bottom stream 40 which is recycled to column 200 (optionally as reflux), and a top stream 39 which is fed via a side inlet to column 220.

Column 220 is maintained at a pressure of from 0.1 to 35 bara. A side draw stream 44 from column 220 is fed to phase separator 260 to remove additional water from the system via aqueous stream 46 which is recycled to the column 200 (optionally as reflux). The organic stream 45 from separator 260 (now depleted of water) is recycled to column 220, optionally below the side draw for stream 44. The residual water forms an azeotropic mixture with the remaining propanol and is distilled from the top of column 220 via stream 41, condensed and a portion refluxed via stream 43 with the balance taken as a tops product via stream 42 along with any remaining methanol and other volatile organic compounds. The remaining butanol and nonvolatile organic compounds, depleted of water, are removed as a column 220 bottoms product via stream 47 to a reboiler, with vapor returned to the column via stream 48 and the balance taken from the column as stream 49. Stream 49 has a color factor greater than 15. Stream 49 is fed to distillation column 230, maintained at pressure of from 0.02 to 35 bara. A bottoms stream 53 is fed to the column reboiler, vapor is returned to column 230 via stream 54, and a purge is drawn off as stream 55, comprising butanol and nonvolatile organic compounds and having a color factor greater than 15. A top stream 50, comprising pure butanol with a color factor less than 15, is condensed, a portion refluxed via stream 52 and the balance drawn off from column 230 as a tops product via stream 51.

The following Examples demonstrate the present invention and its capability for use. The invention is capable of other and different embodiments, and its several details are capable of modifications in various apparent respects, without departing from the scope and spirit of the present invention. Accordingly, the Examples are to be regarded as illustrative in nature and not as restrictive. All percentages are by weight unless otherwise indicated.

### EXAMPLE 1

This experiment demonstrates the embodiment of the present process involving three distillation columns and two phase separators, i.e. evaluating the top, side and bottom streams of distillation column 110, and the top and bottom streams of column 120 of FIG. 1. A feedstock comprising 33% water, 32% methanol, 2% propanol and 33% butanol was fed to a side inlet of a distillation column, such as column 110, maintained at atmospheric pressure. The top stream corresponding to stream 10 of FIG. 1 comprised 4% water, 96% methanol, 0% propanol, 0% butanol, had a temperature of 65° C. and a color factor greater than 30. The side stream corresponding to stream 12 of FIG. 1 comprised 91.5% water, 0.8% methanol, 0.9% propanol and 6.8% butanol and had a temperature of about 92° C. The bottom stream corresponding to stream 17 of FIG. 1 comprised 0.05% water, 0.00% methanol, 0.01% propanol, 99.94% butanol and had a temperature of about 117° C. and color factor greater than 30.

The bottoms stream was collected and then fed to a side inlet of a distillation column, such as column 120, maintained at atmospheric pressure. The top stream from this column, corresponding to stream 19 of FIG. 1, comprised 0.016% water, 0% methanol, 0.008% propanol, 99.98% butanol and had a temperature of about 117° C. and color factor less than 15. The bottom stream corresponding to stream 22 of FIG. 1 comprised 0% water, 0% methanol, 0% propanol, 72% butanol and had a temperature of about 120° C. and a color factor greater than 30.

### EXAMPLE 2

This experiment demonstrates the embodiment of the present process involving a second distillation column such as column 210 of FIG. 2. A feedstock comprising 36% water, 10% methanol, 7% propanol and 47% butanol was fed to a side inlet of a distillation column, such as column 210, maintained at atmospheric pressure. The top stream corresponding to stream 34 of FIG. 2 comprised 6.4% water, 85.5% methanol, 6.5% propanol, and 1.6% butanol and had a temperature of about 66° C. The bottom stream corresponding to stream 36 of FIG. 2 comprised 41.8% water, 0.5% methanol, 4.9% propanol, and 51.0% butanol and had a temperature of about 92° C. The organic portion of this bottom stream, i.e. after treatment in a phase separator such as separator 250 of FIG. 2, can be further treated using distillation columns 220 and 230 of FIG. 2, or distillation columns 110 and 120 of FIG. 1. The configuration of FIG. 2 would be used in the event that recovery of methanol is desired; otherwise the FIG. 1 configuration is appropriate.

All patents, patent applications, test procedures, priority documents, articles, publications, manuals, and other documents cited herein are fully incorporated by reference to the extent such disclosure is not inconsistent with this invention and for all jurisdictions in which such incorporation is permitted.

When numerical lower limits and numerical upper limits are listed herein, ranges from any lower limit to any upper limit are contemplated.

While the illustrative embodiments of the invention have been described with particularity, it will be understood that various other modifications will be apparent to and may be readily made by those skilled in the art without departing from the spirit and scope of the invention. Accordingly, it is not intended that the scope of the claims hereof be limited to the examples and descriptions set forth herein but rather that the claims be construed as encompassing all the features of patentable novelty which reside in the present invention, including all features which would be treated as equivalents thereof by those skilled in the art to which the invention pertains.

1. A process for recovering butanol from a mixture comprising water, methanol, propanol, butanol and optionally other organic compounds comprising the steps of:
a) feeding a mixture comprising water, methanol, propanol, butanol and optionally other organic compounds to a first distillation column maintained at controlled conditions via a side inlet whereby a bottom fraction stream of the first distillation column comprises water, and a top fraction stream of the first distillation column comprises methanol, propanol, butanol, remaining water and other organic compounds,
b) optionally feeding the top fraction stream of step a) to a first phase separator, whereby a bottom stream of the first phase separator comprises water, and a top stream of the first phase separator comprises methanol, propanol, butanol, remaining water and other organic compounds,
c) feeding the top fraction stream of step a) or top stream of step b) to a second distillation column maintained at controlled conditions via a side inlet, whereby a side fraction stream of the second distillation column comprises propanol, butanol, water and other organic compounds, a top fraction stream of the second distillation column comprises methanol, propanol, volatile organic compounds and water, and a bottom fraction stream of the second distillation column comprises butanol and nonvolatile organic compounds,
d) feeding said side fraction stream of step c) to a second phase separator, whereby a top organic phase stream and a bottom aqueous phase stream are created,
e) feeding said organic phase stream of step d) to the second distillation column below the outlet for the side fraction stream of the second distillation column,
f) feeding said aqueous phase stream of step d) and bottom stream of step b) to the first distillation column,
g) feeding said bottom fraction stream of step c) comprising butanol and nonvolatile organic compounds to a third distillation column maintained at controlled conditions, whereby a bottom fraction stream of the third distillation column comprises butanol and nonvolatile organic compounds, and a top fraction stream of the third distillation column comprises from 95 to about 100 wt. % butanol, and
h) recovering said butanol from the top fraction stream of step g).

2. The process of claim 1 wherein at least about 96 wt. % of the top fraction stream of step g) recovered in step h) is butanol.

3. The process of claim 1 or claim 2 wherein the top fraction stream of step g) recovered in step h) has a color factor of less than 15.

4. The process of claim 1 wherein the feed mixture of step a) comprises from about 50 to about 100% water, from about 0.1 to about 15% methanol, from about 0.1 to about 5% propanol, and from about 0.1 to about 30% butanol.

5. The process of claim 1 wherein the controlled conditions of the first distillation column include a pressure from 0.1 to 450 bara

6. The process of claim 1, wherein the controlled conditions of the second distillation column include a pressure from 0.1 to 35 bara.

7. The process of claim 1 wherein the controlled conditions of the third distillation column include a pressure from 0.01 to 35 bara.

8. The process of claim 5 wherein the controlled conditions of the distillation columns include a pressure 1.0±0.2 bara.

9. A process for recovering butanol from a mixture comprising water, methanol, propanol, butanol and optionally other organic compounds comprising the steps of:
a) feeding a mixture comprising water, methanol, propanol, butanol and other organic compounds to a first distillation column maintained at controlled conditions via a side inlet whereby a bottom fraction stream of the first distillation column comprises water, and a top fraction stream of the first distillation column comprises methanol, propanol, butanol, remaining water and other organic compounds,
b) feeding the top fraction stream of step a) to a first phase separator, whereby a bottom aqueous phase stream and a top organic phase stream are created,
c) feeding said aqueous phase stream of step b) to the first distillation column,
d) feeding said organic phase stream of step b) to a second distillation column maintained at controlled conditions via a side inlet whereby a bottom fraction stream of the second distillation column comprises propanol, butanol, water and nonvolatile organic compounds, and a top fraction stream of the second distillation column comprises methanol and volatile organic compounds,
e) feeding the bottom fraction stream of step d) to a second phase separator, whereby a bottom aqueous phase stream and a top organic phase stream are created,
f) feeding said aqueous phase stream of step e) to the first distillation column,
g) feeding said organic phase stream of step e) to a third distillation column maintained at controlled conditions via a side inlet whereby a bottom fraction stream of the third distillation column comprises butanol and nonvolatile organic compounds, a top fraction stream of the third distillation column comprises propanol, butanol, water and volatile organic compounds, and a side fraction stream of the third distillation column comprises propanol, butanol and water,
h) feeding the side fraction stream of step g) to a third phase separator, whereby a bottom aqueous phase stream and a top organic phase stream are created,
i) feeding the top organic phase stream of step h) via a side inlet to the third distillation column,
j) feeding the bottom aqueous phase stream of step h) to the first distillation column,
k) feeding said bottom fraction stream of step g) comprising butanol and nonvolatile organic compounds to a fourth distillation column maintained at controlled conditions, whereby a bottom fraction stream of the fourth distillation column comprises butanol and nonvolatile organic compounds, and a top fraction stream of the fourth distillation column comprises from 95 to about 100 wt. % butanol, and
l) recovering said butanol from the top fraction stream of step k).

10. The process of claim 9 wherein at least about 96 wt. % of the top fraction stream from step k) recovered in step l) is butanol.

11. The process of claim 9 wherein the top fraction stream from step k) recovered in step l) has a color factor of less than 15.

12. The process of claim 9 wherein the feed mixture of step a) comprises from about 50 to about 100% water, from about 0.1 to about 15% methanol, from about 0.1 to about 5% propanol, and from about 0.1 to about 30% butanol.

13. The process of claim 9 wherein the controlled conditions of the first distillation column include a pressure from 0.1 to 450 bara.

14. The process of claim 9 wherein the controlled conditions of the second distillation column include a pressure from 0.2 to 50 bara.

15. The process of claim 9 wherein the controlled conditions of the third distillation column include a pressure from 0.1 to 35 bara.

16. The process of claim 9 wherein the controlled conditions of the fourth distillation column include a pressure from 0.02 to 35 bara.

17. The process of claim 9 wherein the controlled conditions of the distillation columns include a pressure 1.0±0.2 bara.

18. The process of claim 9 wherein the controlled conditions of the first distillation column include a pressure from 0.2 to 3 bara.

19. The process of claim 9 wherein the controlled conditions of the second distillation column include a pressure from 0.2 to 2 bara.

20. The process of claim 9 wherein the controlled conditions of the third distillation column include a pressure from 0.1 to 2 bara.

21. The process of claim 9 wherein the controlled conditions of the fourth distillation column include a pressure from 0.1 to 2 bara.

22. The process of claim 9 wherein the top fraction stream of the second distillation column of step d) is fed to a methanol recovery system.

23. The process of claim 1 wherein the side fraction stream draw-off location of step c) is below the feed location of step c) on the second distillation column.

24. The process of claim 23 wherein the side fraction stream is maintained at less than 100° C.

25. The process of claim 9 wherein the side fraction stream draw-off location of step g) is below the feed location of step g) on the third distillation column.

26. The process of claim 25 wherein the side fraction stream is maintained at less than 100° C.
